(19) United States
(12) Patent Application Publication
(10) Pub. No.: US 2022/0038430 A1
(43) Pub. Date: Feb. 3, 2022

(54) DIRECT API INTEGRATIONS IN PATIENT CARE MANAGEMENT

(71) Applicant: International Business Machines Corporation, Armonk, NY (US)

(72) Inventors: Patrick Arthur Fagan, Naas (IE); Brian Lavin, Naas (IE); LUKE SCOLLAN, Bettystown (IE)

(21) Appl. No.: 16/940,813

(22) Filed: Jul. 28, 2020

(51) Int. Cl. H04L 29/06; G16H 10/60

(52) U.S. Cl. CPC H04L 63/0272; G16H 10/60; H04L 63/0807; H04L 67/42

(57) ABSTRACT

Managing healthcare services by exposing multiple public and private APIs from various entities while protecting them from unauthorized access to applications. Cloud-based healthcare systems are accessed via VPN (virtual private network) connections to directly integrated APIs for interfacing with various architectures, such as consolidated clinical document architecture (CCDA), providing secure access to electronic medical records, electronic health records, and personal health records.

### BACKGROUND

The present invention relates generally to the field of patient care management systems, and more particularly to integration of public and private APIs in healthcare systems.

Patient care management systems involve a set of activities intended to improve patient care and reduce the need for medical services by enhancing coordination of care, eliminating duplication, and helping patients and caregivers more effectively manage health conditions. These efforts have demonstrated potential to improve quality and control costs for patients with complex conditions. Cloud-based care management provides individualized care plans that manage the whole person and engage the community. Relying on secure cloud-based systems, governments can collaborate across departments and agencies, bridge the gap between community and care providers, and automate care management workflows.

A server-side web API (application programming interface) is a programmatic interface consisting of publicly exposed endpoints to a defined request-response message system, typically expressed in JSON (JavaScript object notation) or XML (extensible markup language), which is exposed via the web as an online programming interface. An API enables interaction between data, applications, and devices delivering data and facilitating connectivity between devices and programs. Mashups are web applications which combine the use of multiple server-side web APIs. Webhooks are server-side web APIs that take input as a uniform resource identifier (URI) that is designed to be used as a remote named pipe or a callback such that the server acts as a client to dereference the provided URI and trigger an event on another server that handles the event providing a form of peer-to-peer IPC (inter-process communication).

A private API is an application programming interface whose application is hosted by in-house developers. Private APIs serve as front-end interfaces for back-end data and application functions. The private API provides, for example, a point of entry for developers and/or contractors developing the application functions. A protected API is an exposed public or private API accessible only to authenticated users.

Webhooks in web development facilitate a method of augmenting or changing the behavior of a web page or a web application with custom callbacks. These callbacks may be maintained, modified, and managed by third party users and/or developers who may not necessarily be affiliated with the originating website or application. Webhooks are essentially user-defined HTTP (hypertext transfer protocol) callbacks. They are usually triggered by some event, such as pushing code to a repository or a comment being posted to a blog. When the target event occurs, the source site sends an HTTP request to the uniform resource locator (URL) configured for the webhook. Users can configure webhooks to cause events on one website to invoke behavior on another website. Common uses of webhooks are to trigger builds with continuous integration systems or to notify bug tracking systems. Because webhooks use HTTP, they can be integrated into web services without adding new infrastructure.

A consolidated clinical document architecture (CCDA) is a complete architecture used to create documents and template methodologies for medical documents. The primary function of the CCDA is to standardize the content and structure for clinical care summaries.

### SUMMARY

According to an aspect of the present invention, there is a method, computer program product and/or system that performs the following operations (not necessarily in the following order): (i) accessing a private application programming interface (API) of a first entity using a first dedicated virtual private network (VPN); (ii) accessing a public API of a second entity; (iii) establishing a secure gateway with integration points for interfacing with multiple type of computing architectures; (iv) integrating the private API and the public API with a set of published APIs available at the secure gateway; (v) authenticating a user for access to the secure gateway; and (vi) providing access to the authenticated user for confidential records using the private API integrated into the gateway and via the first dedicated VPN.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a screenshot view generated by the first embodiment system;

FIG. 5 is a first diagram of a system according to an embodiment of the present invention;

FIG. 6 is a second diagram of a system according to an embodiment of the present invention;

FIG. 7 is a third diagram of a system according to an embodiment of the present invention; and

FIG. 8 is a tree diagram according to an embodiment of the present invention.

### DETAILED DESCRIPTION

Some embodiments of the present invention are directed to managing web content by exposing multiple public and private APIs from various entities while protecting them from unauthorized access to applications. Cloud-based healthcare systems are accessed via VPN (virtual private network) connections to directly integrated APIs for interfacing with various architectures, such as consolidated clinical document architecture (CCDA), providing secure access to electronic medical records, electronic health records, and personal health records.

This Detailed Description section is divided into the following subsections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

A “storage device” is hereby defined to be anything made or adapted to store computer code in a manner so that the computer code can be accessed by a computer processor. A storage device typically includes a storage medium, which is the material in, or on, which the data of the computer code is stored. A single “storage device” may have: (i) multiple discrete portions that are spaced apart, or distributed (for example, a set of six solid state storage devices respectively located in six laptop computers that collectively store a single computer program); and/or (ii) may use multiple storage media (for example, a set of computer code that is partially stored in as magnetic domains in a computer's non-volatile storage and partially stored in a set of semiconductor switches in the computer's volatile memory). The term “storage medium” should be construed to cover situations where multiple different types of storage media are used.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

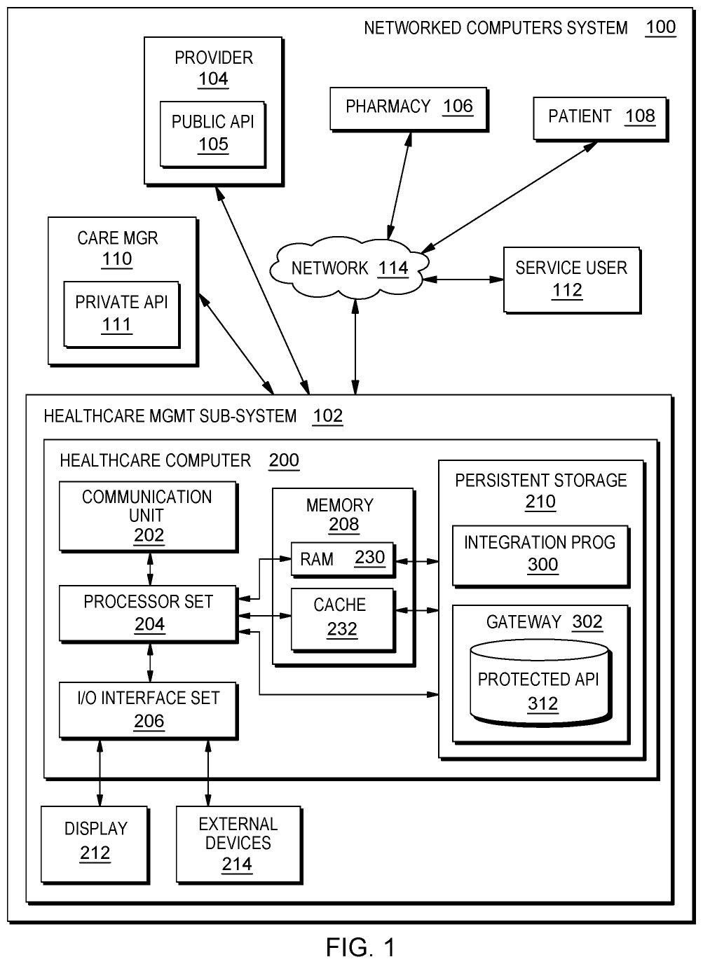

As shown in FIG. 1, system 100 is an embodiment of a hardware and software environment for use with various embodiments of the present invention. System 100 includes: healthcare management subsystem 102 (sometimes herein referred to, more simply, as subsystem 102); healthcare provider subsystem 104; public API 105; pharmacy subsystem 106; patient subsystem 108; care manager subsystem 110; private API 111; web user subsystem 112; and communication network 114. healthcare management subsystem 102 includes: healthcare computer 200; communication unit 202; processor set 204; input/output (I/O) interface set 206; memory 208; persistent storage 210; display 212; external device(s) 214; random access memory (RAM) 230; cache 232; integration program 300; secure gateway module 302; and protected API store 312.

Subsystem 102 may be a laptop computer, tablet computer, netbook computer, personal computer (PC), a desktop computer, a personal digital assistant (PDA), a smart phone, or any other type of computer (see definition of “computer” in Definitions section, below). Program 300 is a collection of machine readable instructions and/or data that is used to create, manage and control certain software functions that will be discussed in detail, below, in the Example Embodiment subsection of this Detailed Description section.

Subsystem 102 is capable of communicating with other computer subsystems via communication network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client subsystems.

Subsystem 102 is shown as a block diagram with many double arrows. These double arrows (no separate reference numerals) represent a communications fabric, which provides communications between various components of subsystem 102. This communications fabric can be implemented with any architecture designed for passing data and/or control information between processors (such as microprocessors, communications and network processors, etc.), system memory, peripheral devices, and any other hardware components within a computer system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for subsystem 102; and/or (ii) devices external to subsystem 102 may be able to provide memory for subsystem 102. Both memory 208 and persistent storage 210: (i) store data in a manner that is less transient than a signal in transit; and (ii) store data on a tangible medium (such as magnetic or optical domains). In this embodiment, memory 208 is volatile storage, while persistent storage 210 provides nonvolatile storage. The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202 provides for communications with other data processing systems or devices external to subsystem 102. In these examples, communications unit 202 includes one or more network interface cards. Communications unit 202 may provide communications through the use of either or both physical and wireless communications links. Any software modules discussed herein may be downloaded to a persistent storage device (such as persistent storage 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. I/O interface set 206 also connects in data communication with display 212. Display 212 is a display device that provides a mechanism to display data to a user and may be, for example, a computer monitor or a smart phone display screen.

In this embodiment, program 300 is stored in persistent storage 210 for access and/or execution by one or more computer processors of processor set 204, usually through one or more memories of memory 208. It will be understood by those of skill in the art that program 300 may be stored in a more highly distributed manner during its run time and/or when it is not running. Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

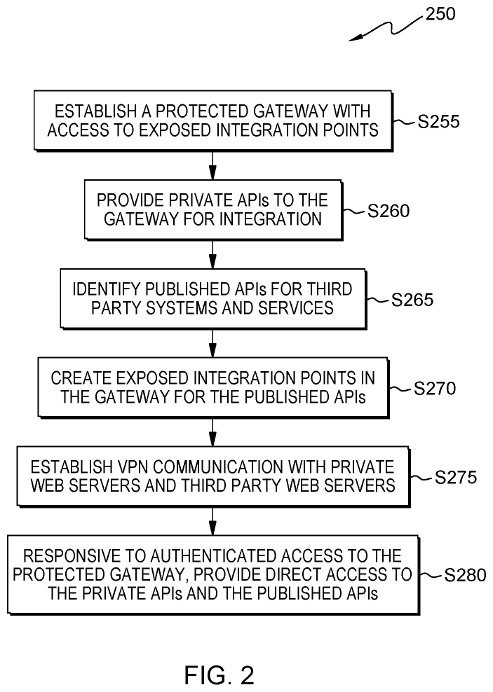

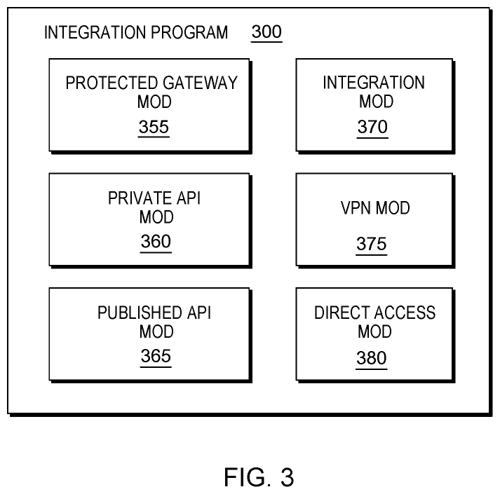

As shown in FIG. 1, system 100 is an environment in which an example method according to the present invention can be performed. As shown in FIG. 2, flowchart 250 shows an example method according to the present invention. As shown in FIG. 3, program 300 performs or control performance of at least some of the method operations of flowchart 250. This method and associated software will now be discussed, over the course of the following paragraphs, with extensive reference to the blocks of FIGS. 1, 2 and 3.

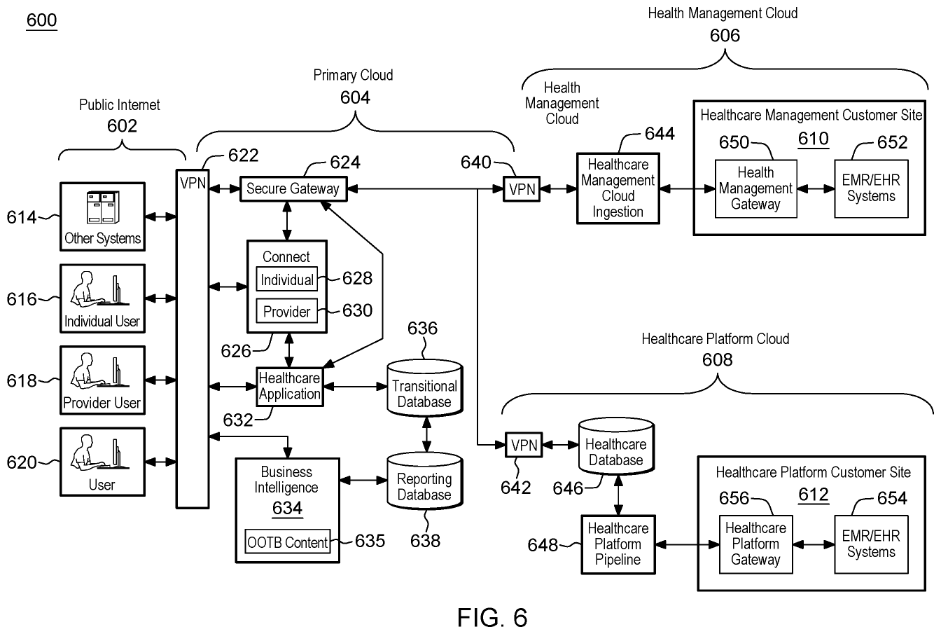

Processing begins at operation 5255, where protected gateway module (“mod”) 355 establishes a protected gateway with access to exposed integration points. In this example, gateway 302 is established in healthcare management sub-system 102 that contains and exposes external-facing services via protected API store 312 for access via network 114, which is considered in this example to be an untrusted, larger network. Alternatively, the gateway is established in a secure physical or logical subnetwork that contains and exposes external-facing services to a public network, such as public internet 602 (FIG. 6). Some embodiments of the present invention establish the gateway with access limited to virtual private network (VPN) access. Some embodiments of the present invention establish a single firewall for securing access to services provided by a back end private network. Other embodiments of the present invention establish a dual firewall infrastructure for securing the back end private network.

Processing proceeds to operation 5260, where private API mod 360 provides private API's to the gateway for integration. In this example, access to private API 111 of care manager sub-system 110 is provided to gateway 302. Further, in this example, access to private API 111 is stored in protected API store 312 for access via gateway 302. As shown in FIG. 1, persistent storage 210, within computer 200, uses integration program 300 along with protected API storage 312 within gateway 302 to provide the private API's. Alternatively, private APIs are identified, and access information is obtained for integration of the private APIs into the established gateway.

Processing proceeds to operation 5265 where published API mod 365 identifies published API's for third party systems and services. In this example, access to public API 105 of provider sub-system 104 is provided to gateway 302. Further, in this example, access to public API 105 is stored in protected API store 312 for access via gateway 302. As shown in FIG. 1, persistent storage 210, within computer 200, uses integration program 300 along with protected API storage 312 within gateway 302 to provide the public API's. Alternatively, public APIs are identified, and access information is obtained for integration of the public APIs into the established gateway.

Processing proceeds to operation 5270 where integration mod 370 creates exposed integration points in the gateway for published API's. In this example, published APIs such as APIs stored in protected API store 312 are exposed to network 114 via integration points of gateway 302. Alternatively, the integration points are exposed to a public network for access to private API 111 and public API 105. According to some embodiments of the present invention access to the exposed integrations points is limited to VPN access the gateway.

Processing proceeds to operation 5275 where VPN mod 375 establishes VPN communications with private web services and third-party web servers. On the backend of the gateway access to private web services and third-party web servers is limited to VPN access. By requiring VPN access, additional security is made available to sensitive data provided through the various exposed APIs at the gateway integration points. Examples of VPN security are further illustrated in FIG. 6 at VPN 640 and VPN 642 where communications to healthcare management cloud 606 and healthcare platform cloud 608 is restricted to VPN communications.

Processing concludes at operation 5280 where direct access mod 380, responsive to authenticated access to the protected gateway, provides direct access to the private API's and the published API's. In this example, access to protected APIs, private APIs, and/or public APIs is provided via gateway 302 upon authenticated access by, for example, service user sub-system 112 communicating over network 114. Some embodiments of the present invention provide integration points in the gateway for APIs subscribed to by the accessing user. Upon storing the API as an integration point in the gateway, updates to the API address are submitted and updated at the gateway as shown in FIG. 8.

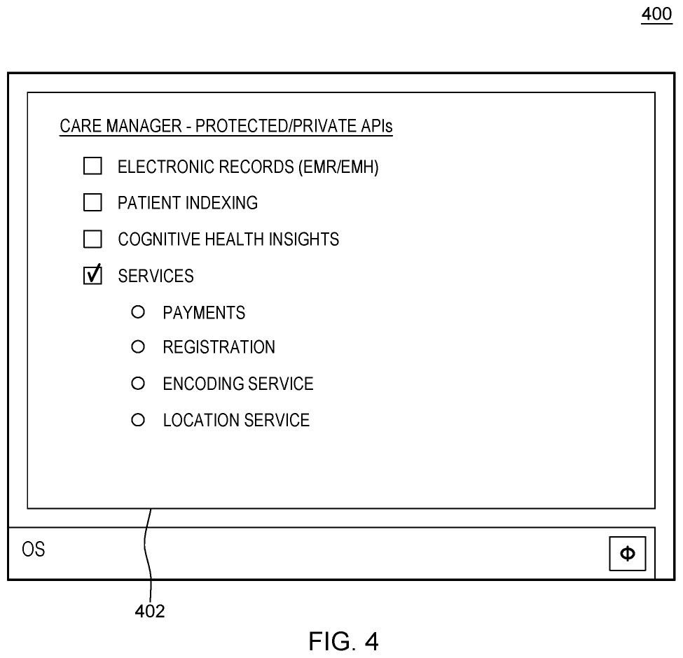

Referring now to FIG. 4, screenshot 400 provides an example display 402 of a healthcare manager system providing user access to protected and/or private APIs with user selectable categories including: (i) electronic records such as EMRs and EMHs; (ii) patient indexing such as a master patient index or sources of clinical and non-clinical data available as part of a care management system; (iii) cognitive health insights where big data generated through the care management process is processed to provide, for example key information for managing healthcare of patients; and (iv) standard healthcare services such as payments processing, registration and registries, health term encoding services, and locations services.

### III. Further Comments and/or Embodiments

Some embodiments of the present invention recognize the following facts, potential problems and/or potential areas for improvement with respect to the current state of the art: (i) cloud-based healthcare solutions are typically part of a larger ecosystem of software systems used by care managers and patients; and (ii) cloud-based healthcare solutions needs to include: (a) packaged integrations that include: (1) large company offerings (for example, a company that provides a secure, cloud-based platform, longitudinal EHR (electronic health record) data, and analytic tools to help providers and life sciences companies find new connections among previously siloed healthcare data, and (2) third party offerings (for example, an online company that operates an online payment system that supports online money transfers and serves as an electronic alternative to traditional paper payment methods), and (b) integration mechanisms such as APIs (application programming interfaces) to allow customers/partners to build integrations with other systems that recognize: (1) the type and number of other systems vary by customer, and (2) there is a broad spectrum of data/workflow integration requirements, that is, a large number of integration points, or APIs, need to be exposed.

Some embodiments of the present invention recognize that healthcare management features will be exposed via a set of various external and internal APIs which can used by different systems to integrate and interoperate with cloud-based healthcare management.

Some embodiments of the present invention recognize that healthcare systems should: (i) collect, link, and combine data from EHR and EMPI (enterprise master patient index) systems to identify and transfer populations and/or cohorts for care management; (ii) act as a source of selected clinical and/or non-clinical data in care management; (iii) import care management updates to enhance patient information and feedback to source systems; and (iv) invoke cognitive computing and/or AI (artificial intelligence) systems to process large amounts of data generated through the care management process and send insights to help care managers identify key information about patients without manually going through all the data.

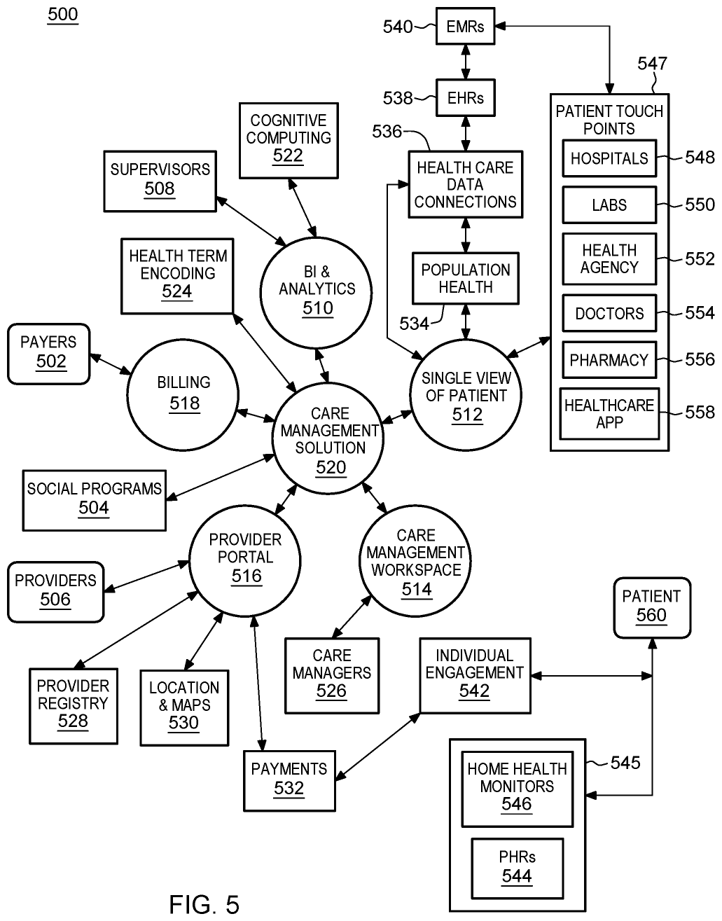

FIG. 5, diagram 500 is system of integration and interoperability flows that includes the following: payers sub-system 502; social programs node 504; providers sub-system 506; supervisors block 508; BI (business intelligence) and analytics node 510; single view of patient node 512; care management workspace node 514; provider portal node 516; billing node 518; care management solution node 520; cognitive computing node 522; health term encoding block 524; care managers node 526; provider registry block 528; location and maps block 530; payments block 532; population health block 534; healthcare data connections block 536; EHRs block 538; EMRs block 540; individual engagement block 542; PHRs (professional in human resources) block 544; patient resources 545; home health monitors block 546; Patient touch points set 547; hospitals point 548; labs point 550; health agency point 552; doctors point 554; pharmacy point 556; healthcare application point 558; and patient sub-system 560.

Again referring to FIG. 5, system diagram 500, according to an embodiment of the present invention, also includes the following processes: (i) cohort referrals and client data for care management communicating between social programs 504 and care management solution node 520; (ii) cohort referrals and client data (including EMPI) communications between population health 534 and single view of patient node 512; (iii) payment to providers for their services communications between payments 532 and provider portal node 516; (iv) allow patients to pay for provider services through the platform by communicating between individual engagement 542 and payments 532; (v) clinical and non-clinical data exchange communications between healthcare data connections 536 and single view of patient node 512; (vi) import of PHR data and sharing patient's view from the care management service in an individual engagement platform with communications between individual engagement 542 and single view of patient node 512; (vii) upload of PHRs to an online platform and integration with care management process with communications between individual engagement 542 and patient resources 545; and (viii) communications by patient touch points set 547 with single view of patient node 512.

FIG. 6, diagram 600 is a system of logical data flows that includes the following: public internet 602; primary cloud 604; health management cloud 606; healthcare platform cloud 608; health management customer site 610; healthcare platform customer site 612; other systems block 614; individual user block 616; provider user block 618; service user block 620; VPN (virtual private network) block 622; secure gateway block 624; connect block 626 (which includes individual block 628 and provider block 630); healthcare application block 632; business intelligence block 634 (which includes OOTB (out-of-the-box) content block 635; transactional database 636; reporting database 638; VPN block 640; VPN block 642; health management cloud ingestion block 644; healthcare database 646; healthcare platform pipeline block 648; health management gateway block 650; EMR (electronic medical records)/EHR (electronic health record) systems block 652; EMR/EHR systems block 654; and healthcare platform gateway block 656.

Some embodiments of the present invention may include one, or more, of the following operations, features, characteristics and/or advantages: (i) provides a set of private APIs that form the basis of the integrations with large company offerings; (ii) uses published APIs to integrate with third parties; and (iii) uses a large set of protected APIs to enable direct integrations including: (a) inbound notes—get, create, update, delete a note or a touchpoint, (b) inbound alerts—create or update or delete an alert, (c) inbound individuals—can be used to create demographics data for an individual, (d) inbound phone—get, create, update, delete a phone number, (e) inbound address—get, create, update, delete an address, (f) inbound email—get, create, update, delete an email address, (g) inbound assessment score—create, delete an assessment score, (h) inbound custom data—get, create, update, delete data for the specified custom data type, (i) inbound referral—create a referral, (j) inbound consent—create a consent record, (k) outbound demographics—notify about demographic changes, and (l) outbound care plan—notify to get a care plan summary.

Some embodiments of the present invention may include one, or more, of the following operations, features, characteristics and/or advantages: (i) includes RESTful (representational state transfer) Java APIs; (ii) is secure using VPN in addition to authorization checks; (iii) uses standards where applicable (for example, FHIR (fast healthcare interoperability resources)); and (iv) webhooks are used for outbound connections. (Note: the term(s) “RESTFUL,” “FHIR,” and/or “JAVA” may be subject to trademark rights in various jurisdictions throughout the world and are used here only in reference to the products or services properly denominated by the marks to the extent that such trademark rights may exist.)

Some embodiments of the present invention may include one, or more, of the following operations, features, characteristics and/or advantages: (i) EMR (electronic medical record) and/or EHR systems share records across different health care settings which also act as source clinical and non-clinical data for care management process; (ii) packaged software used for other business functions (that is, ERP (enterprise resource planning) and case management) to import data enables internal business processes custom mobile apps to integrate care management data in care manager or patient engagement; (iii) uses service-based systems to enable processes that cover lookup for standard terms for clinical data such as allergies and medications; (iv) provides map and location services for patients and care services location assessment; (v) supports payment services that provide a secure platform for payments; and (vi) uses open SaaS (software as a service) based care providers registry to allow patients to select appropriate care providers.

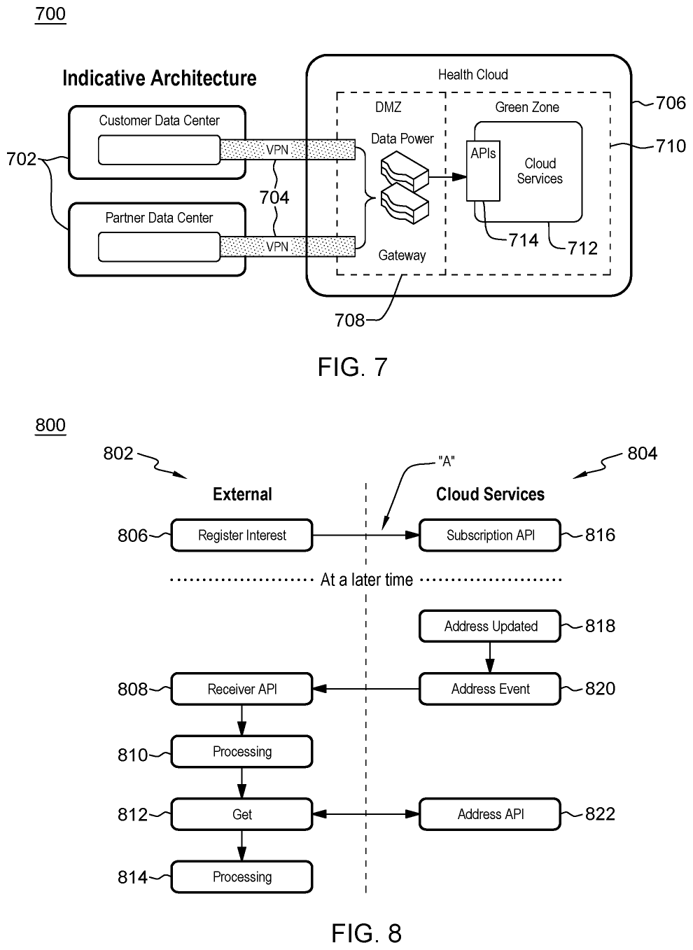

FIG. 7, diagram 700 is an indicative architecture system that performs security and authentication. Diagram 700 includes the following: partner data centers 702; VPNs 704; health cloud 706; DMZ (screened subnet) gateway 708; green zone 710; and cloud services 712, which includes APIs 714.

Some embodiments of the present invention may include one, or more, of the following operations, features, characteristics and/or advantages: (i) in regard to security, a dedicated VPN is required to access the health care manager APIs; and (ii) in regard to authentication, the REST API services require authentication before access to the APIs is allowed and includes: (a) logging in with the API URL (uniform resource locator) and retrieving the authentication token, and (b) all API requests must contain additional header parameters including the authentication token.

FIG. 8, table 800 shows the approach to outbound APIs and includes the following: external side 802; cloud services side 804; register interest block 806; receiver API block 808; processing block 810; get block 812; processing block 814; subscription API block 816; address updated block 818; address event block 820; and address API block 822.

Some embodiments of the present invention recognize the following approach concerning outbound APIs: (i) use webhooks to broadcast thin events as needed; (ii) key characteristics include: (a) security—no PHI (protected health information) included in event message; and (b) stale data—even if event messages arrive out of order, the receiver will still get the correct data when they request it; and (iii) the registration message includes a pointer to the receiver API (see “A” in FIG. 8).

Some embodiments of the present invention recognize a method for enabling one or more direct integration by utilizing a large set of protected application programming interfaces (APIs), comprising: (i) receiving one or more sets of private APIs wherein: (a) the received one or more sets of private APIs are the basis of the direct integration, (b) a dedicated virtual private network (VPN) and a plurality of authentication checks are utilized as a security requirement to access the received one or more sets of private APIs, (c) the authentication check includes a user authenticating a user identity before accessing the received one or more sets of private APIs, (d) the user submits an API request for access to the received one or more sets of private APIs, and (e) the API request includes one or more additional header parameters and an authentication token; (ii) integrating a plurality of published APIs with one or more third parties; and (iii) generating the one or more direct integrations with the large set of protected APIs.

Some embodiments of the present invention may include one, or more, of the following operations, features, characteristics and/or advantages: (i) combinations of a set of APIs (based on standards), a number of technical designs which govern the implementations of the APIs and the technical mechanism for the consumer of the APIs to connect; and (ii) there is no overlap about the combination of techniques to implement the integration itself.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method comprising:
accessing a private application programming interface (API) of a first entity using a first dedicated virtual private network (VPN);
accessing a public API of a second entity;
establishing a secure gateway with integration points for interfacing with multiple type of computing architectures;
integrating the private API and the public API with a set of published APIs available at the secure gateway;
authenticating a user for access to the secure gateway; and
providing access to the authenticated user for confidential records using the private API integrated into the gateway and via the first dedicated VPN.

2. The computer-implemented method of claim 1, further comprising:
receiving an authentication request for the secure gateway from the user over a second dedicated VPN.

3. The computer-implemented method of claim 1, further comprising:
receiving an API request from the authenticated user to access the private API, the API request including header parameters and an authentication token; and
submitting the authentication token to the first entity over the first dedicated VPN.

4. The computer-implemented method of claim 1, wherein a first integration point for the private API includes a dedicated VPN connection for interfacing with a consolidated clinical document architecture (CCDA).

5. The computer-implemented method of claim 4, wherein the confidential records include electronic medical records for a first patient.

6. The computer-implemented method of claim 1, wherein access to the public API is available to the authenticated user based on a subscription to a service provided by the second entity, the subscription associated with the authenticated user.

7. A computer program product comprising:
a set of storage device(s); and
computer code stored collectively in the set of storage device(s), with the computer code including data and instructions to cause a processor(s) set to perform at least the following operations:
accessing a private application programming interface (API) of a first entity using a first dedicated virtual private network (VPN);
accessing a public API of a second entity;
establishing a secure gateway with integration points for interfacing with multiple type of computing architectures;
integrating the private API and the public API with a set of published APIs available at the secure gateway;
authenticating a user for access to the secure gateway; and
providing access to the authenticated user for confidential records using the private API integrated into the gateway and via the first dedicated VPN.

8. The computer program product of claim 7, further comprising computer code including data and instructions to cause the processor(s) set to perform the following operation:
receiving an authentication request for the secure gateway from the user over a second dedicated VPN.

9. The computer program product of claim 7, further comprising computer code including data and instructions to cause the processor(s) set to perform the following operations:
receiving an API request from the authenticated user to access the private API, the API request including header parameters and an authentication token; and
submitting the authentication token to the first entity over the first dedicated VPN.

10. The computer program product of claim 7, wherein the confidential records include electronic medical records for a first patient.

11. The computer program product of claim 7, wherein access to the public API is available to the authenticated user based on a subscription to a service provided by the second entity, the subscription associated with the authenticated user.

12. A computer system comprising:
a processor(s) set;
a set of storage device(s); and
computer code stored collectively in the set of storage device(s), with the computer code including data and instructions to cause the processor(s) set to perform at least the following operations:
accessing a private application programming interface (API) of a first entity using a first dedicated virtual private network (VPN);
accessing a public API of a second entity;
establishing a secure gateway with integration points for interfacing with multiple type of computing architectures;
integrating the private API and the public API with a set of published APIs available at the secure gateway;
authenticating a user for access to the secure gateway; and
providing access to the authenticated user for confidential records using the private API integrated into the gateway and via the first dedicated VPN.

13. The computer system of claim 12, further comprising computer code causing the processor(s) set to perform the following operation:
receiving an authentication request for the secure gateway from the user over a second dedicated VPN.

14. The computer system of claim 12, further comprising computer code causing the processor(s) set to perform the following operations:
receiving an API request from the authenticated user to access the private API, the API request including header parameters and an authentication token; and
submitting the authentication token to the first entity over the first dedicated VPN.

15. The computer system of claim 12, wherein the confidential records include electronic medical records for a first patient.

16. The computer system of claim 12, wherein access to the public API is available to the authenticated user based on a subscription to a service provided by the second entity, the subscription associated with the authenticated user.
